(19) United States
(12) Patent Application Publication
(10) Pub. No.: US 2002/0041860 A1
(43) Pub. Date: Apr. 11, 2002

(54) Fragranced hydrogel air freshener kits

(76) Inventor: Luz P. Requejo, Racine, WI (US)

Correspondence Address: S.C. JOHNSON & SON, INC., 1525 HOWE STREET, RACINE, WI, 53403-2236, US

(21) Appl. No.: 10/004,688

(22) Filed: Dec. 5, 2001

Related U.S. Application Data
(63) Continuation-in-part of application No. 09/523,005, filed on Mar. 10, 2000.

(51) Int. Cl. A61L 9/00; A61L 9/01

(52) U.S. Cl. 424/76.1

(57) ABSTRACT

Kits for consumer activated hydrogel air fresheners are provided, comprising a gel forming superabsorbent polymer resin, a fragrancing material, and a coloring agent. The kit may take various forms, such as a container in which are packaged the appropriate amounts of resin, fragrance, and colorant, to which a volume of gel forming liquid is to be added. In another form, the kit may comprise a sachet or pad containing the above materials, to which liquid may be added to form a gel air freshener device. Further, a pad or sachet, containing the gel forming resin, and a coloring agent if desired, may be subjected to the controlled continuous feed of a water solution of a fragrance, whereby a long term, consistent fragrancing is obtained.

### RELATED APPLICATION(S)

This is a Continuation-in-Part of U.S. patent application Ser. No. 09/523,005, filed Mar. 10, 2000.

### FEDERALLY SPONSORED RESEARCH OR DEVELOPMENT

Not applicable.

### BACKGROUND OF THE INVENTION

1. Technical Field

The present invention relates to air fresheners comprising a fragrance and a hydrogel composition suitable for preparation and activation by the consumer. The air fresheners so prepared may take a variety of forms, determined by the specific materials provided, from gel compositions which release fragrance by evaporation, to impregnated sachets containing both resin and fragrance, which permit absorption of water, but permit release of their contents only as a vapor, to hydrogel coated cellulosic or non-woven polyolefinic material which is continuously fed with fresh fragrance by controlled liquid release from a reservoir, or spray hydrogel and fragrance coated pads activated by the addition of water.

2. Background

It is known that gel materials may be prepared from a variety of differing polymeric resins. It is also known that such gels may be impregnated with fragrance or deodorant materials. Many such gels have been sold to the public in the form of air fresheners, usually in glass or plastic containers for placement at a location of the consumer's choice.

For example, Kliment, U.S. Pat. No. 4,587,129, teaches that lower alkyl alkenoate based hydrogels may be used for entrapping flavors and fragrances, for use as solid air fresheners or sachets.

Gould et al, in U.S. Pat. No. 3,576,760, disclose preparation of dry powders of water soluble hydroxyalkyl acrylate or methacrylate polymers useful as carriers for fragrances. Such materials as orange oil may be entrapped in the polymer for release upon contact with water.

In U.S. Pat. No. 3,567,118, Shepard et al describe dry products comprising fiber substrates impregnated with hydrophilic gels of (meth)acrylate polymers entrapping fragrance-emitting agents, which release fragrance when wetted.

Martin et al, in U.S. Pat. No. 5,976,503, and Booth, Jr., et al, in U.S. Pat. No. 4,869,407, teach air fresheners including means for active dispensing of fragrance. In Martin et al, the dispensing means employs heat, while Booth, Jr., et al employ a bellows to dispense fragrance from an impregnated paper based wafer disc.

Semoff et al, in U.S. Pat. No. 5,679,334, disclose a transparent gel air freshener containing an ornamental botanical.

Fujiura et al, U.S. Pat. No. 5,904,028, discloses a diffusing device containing a water-swellable gel carrying a fragrance agent. The device comprises a packet having a water permeable portion, and the patent teaches immersing the packet in water containing a fragrance whereby the resin absorbs the fragrance for subsequent release. However, the reference fails to teach the “kit” approach of the present invention, providing a finished air freshener device which comprises a gel formed from a water absorbing, gel-forming resin and at least one fragrance, and a water insoluble container enclosing the gel.

In addition, Pera, in U.S. Pat. No. 4,906,488, discloses compositions comprising liquid-to-soft gels, of monomers, prepolymers, or polymers, referred to collectively as “mers”, having a permeant therein for slow release to the environment. The patent is directed to formation of a solution of “mer” in the permeant, reacting the “mer” without encapsulation, and recovering a liquid product useful in slow-release products.

One problem with such air fresheners is that the fragrance material, or the air freshening component therein, often dissipates or evaporates to some extent before being opened by the consumer, as a result of poor packaging, or exposure to moisture or extreme temperature variation during shipment. Accordingly, one of the objects of the present invention is to provide an air freshener which is activated by the consumer at the time of use, and is thus fresh and complete until such time as it is to be used. Another object of the present invention is to provide consumer activated air fresheners of very low cost, such as may be employed by consumers of any economic status, with the greatest ease of activation.

### SUMMARY OF THE INVENTION

The present invention is directed to a low cost, convenient, consumer activated fragrance releasing device which retains its full fragrance capacity until subjected to the addition of water to cause the reaction of components thereof, thereby forming an expanded gel from which the fragrance is released over a period of time. Thus, the invention is directed to a kit for the preparation of an air freshener device, said kit being capable of long term storage and rapid, convenient activation at the consumer's will.

In its broadest sense, the invention comprises providing to the consumer a package of a dry gel precursor, a fragrancing material, and a colorant if desired, to be combined, by mixing with water, in an appropriate container. In this embodiment of the invention, a dry resinous material, impregnated with colorant, may be mixed with a solid or a liquid fragrance, followed by the addition of a relatively large volume of water. The coloring agent, may of course, be present in the fragrance, or in the water, rather than in the resin, if so desired. Upon stirring or shaking, a colored gel is formed, having the fragrance absorbed therein for release by evaporation over a period of time.

In another embodiment of the invention, a sachet is prepared, comprising a water permeable package of such material as shall permit the passage of liquid to the interior of the package, but shall not permit liquid to flow outwardly. Such materials are known for use in personal care products, such as sanitary napkins and diapers, and permit the passage of liquid in one direction only. This type of material may comprise conically apertured polyethylene film, having small diameter openings designed to permit flow of liquid in one direction, but to close and prohibit back flow of the liquid. This material shall henceforth be referred to herein as having one way permeability to water. Vapors are able to escape from the microscopic openings in the surface of such materials, however, so that if a fragrance is enclosed within the material, the evaporation of such fragrance may occur as desired. While it is desirable that the entire sachet be of the same material, it is possible for only a limited amount of the surface area of the sachet be so characterized, provided that the remaining surface area be impermeable to water. In the sachet may be combined a dry resin, a colorant if desired, and a particulate fragrance. To activate the sachet, the consumer need only immerse it in water, or spray the surface thereof with sufficient water to activate the gel formation of the resin and dissolve the fragrance, if it is water soluble. After being contacted by a sufficient volume of water, the activated sachet may be removed and hung in any convenient location, such as in the home or auto, for the consumer to enjoy its pleasant aroma as the fragrance component thereof is released to the atmosphere. Because the sachet is manufactured of material having one way permeability to water, no excess water absorbed during activation will be released, preventing the loss of any soluble fragrance, and preventing the formation of unsightly stains, puddles, rings, etc.

In still another related embodiment of the invention, a sachet as described above, or a pad of resin and fragrance impregnated or coated material, is placed in a convenient location, such as a dish or bowl, and a controlled amount of water is released upon the surface thereof. By the term pad, applicant refers to a substrate or a cloth like structure of uniform nature, as opposed to a sachet, which comprises an outer cover surrounding separate and distinct internal contents. Such a pad may constitute a woven, e.g. cloth, or non woven fabric, e.g. felt, as well as paper. While the preferred fibrous materials are cellulosic, e.g. cotton and paper, there may be employed other fibers such as nylon, acrylonitrile fibers, polyvinyl chloride fibers, wool, and polyethylene terephthalate fibers. The preferred composite fiber comprises a paper or cardboard like structure.

In this manner, the dry resin is caused to absorb the water and form a gel, which is subsequently contacted by additional water. By control of the rate of addition of liquid to the pad, based upon the evaporation rate of the fragrance mixture from the gel, a consistent rate of fragrance release, and a no-fade dispenser thereof, is attained. In such an embodiment, the fragrance may be in a solid form, mixed with the resin in the pad, or alternatively, may be added in liquid form to the water which is permitted to contact the pad over time. In still a further alternative to this embodiment, the gel itself may be provided to the consumer, rather than a gel coated or impregnated substrate. In such an instance, applicant envisions providing a source of water, or fragrance dissolved in water, said source having a controlled rate of release, at the approximate rate of evaporation of the water absorbed by the gel, so as to obtain a consistent rate of fragrance release. Such controlled rate of release may be obtained, for example, by gravity means, or metering of the water through a channel having holes of the appropriate diameter, or by wicking means.

### DETAILED DESCRIPTION OF THE INVENTION

In the simplest embodiment of the invention, the consumer is provided a kit for the preparation of an air freshener device. The basic components of the kit comprise a measured amount of a dry resinous material which when mixed with water or a suitable gel forming liquid will absorb the water or gel forming liquid to form a gel. While various gel forming resins are known, the preferred materials for the present invention comprise rapidly gel forming materials such as those referred to as superabsorbent polymers. Among such superabsorbent gel forming resins are cross linked sodium polyacrylate, potassium polyacrylate/acrylamide copolymer, polyacrylamide polymer, and Dynasorb Terrasafe, a synthetic rubber based white powder available from Stewart Group. It is to be noted that since the intent of the present invention is to provide a product for home use by the consumer, it is most desirable to utilize materials which gel rapidly and with the least amount of interaction by the consumer. Most preferred among these superabsorbent gel forming resins is cross linked sodium polyacrylate, for reasons of stability, low cost, high liquid absorbency, and ease of use. The superabsorbent resins are characterized by superior absorption and retention of fluids. The gels resulting from such superabsorbent resins retain fluids without wicking, even under stress since the absorption of fluids is based upon a physical-chemical process. Selection of the right superabsorbent polymer or gel forming resin is critical in that this affects the integrity of the gel air freshener formed by the consumer. Highly cross linked sodium polyacrylate is preferred over potassium polyacrylate, or synthetic rubber, since it forms a gel which retains its strength without wicking, with the right selection of fragrance. Potassium polyacrylate/acrylamide copolymer will absorb highly ionic liquid, like sea water, while the synthetic rubber superabsorbent gel forming material Dynasorb Terrasafe will absorb hydrocarbons or oily materials, such as oil based fragrances, but will repel water. Accordingly, suitable gel forming liquids include water and hydrocarbons, the choice of which is dependent upon the superabsorbent gel forming resin to be used.

The gel forming resin is preferably provided as a solid particulate or powder, packaged in a plastic bag, for example, in a sachet such as disclosed previously, characterized as having one way permeability to water, or in a small vial. It is desirable that such packaging be air-tight, so as to limit the undesirable access thereto of water, humidity or air borne moisture. Further, such packaging should preferably be easily openable, economically produced, and ecologically safe for disposal. The size distribution of the gel forming resin, such as sodium polyacrylate, has been found to be critical for the purpose of the present invention. Although superabsorbent polymers are commercially available in different particle size distributions, it has been found that although they are made of the same material and processed the same way, the particle size distribution affects the gelling rate. For example, small particle size sodium polyacrylate gels so rapidly that the consumer may have insufficient time to mix the components thoroughly, resulting in inconsistent release of fragrance from the air freshener prepared. On the other hand, too large a particle size will gel too slowly, resulting in a potential safety and spillage risk to the consumer who is expected to utilize the kit of the present invention. While larger particle size superabsorbent gel forming resin provides a more attractive, and more crystalline appearing product, it has been found that sodium polyacrylate particle sizes above about 1000 microns gel too slowly, and may result in a potential hazard in a do-it-yourself consumer product. As indicated previously, it has also been found that sodium polyacrylate particles below about 100 microns in size gel too rapidly. Accordingly, superabsorbent polymer powder having a particle size range of from about 100 to 900 microns, and preferably from 150 to about 850 microns, constitutes the preferred gel forming resin for the present invention.

As previously indicated, the preferred hydrogel resin for the present invention comprises a polyacrylate polymer, of the type used for superabsorbents. Typical of such resins are Alcosorb G1 acrylic resin, available from Ciba Specialty Chemicals, of Suffolk Va., a cross-linked sodium polyacrylate polymer, having internal cross-linking only, and providing a high degree of swelling and rapid water release. Also available for the present invention, from Ciba, are Alcosorb G5, a double cross-linked polyacrylate polymer having both interior and exterior cross-linking, and Alcosorb AB3C, a copolymer of acrylamide sodium acrylate generally used as a flocculent, having larger particle size, in the range of from 800 to 1200 microns, which yields a large crystal like appearance after absorption of water. An additional suitable resin is sold by Ciba Specialty Chemicals under the trade name Gelling Agent #14, which is a white free flowing powder comprising a cross-linked sodium polyacrylate. It is to be noted that the particle size of such resins will affect the appearance of the finished gel air freshener. Small, powder sized resin will produce a Jello®-like gel, while larger particulate resins will produce a crystal like gel. Similar hydrogel forming resins are also available under various trade-names, such as Horta-Sorb®, from Horticultural Alliance, Inc., of Sarasota, Fla., Cabloc CT™ of Stochausen, Germany, and Hydrosorb™ from The Illinois Marketing Board. Further, the rate of absorbency of the resin is dependent upon the degree of cross-linking and particle size of the superabsorbent, with less cross linkage providing more rapid absorption, and more rapid release of water.

Packaged with the resin in the kit may be a fragrancing material, such as a conventional air freshener fragrance, in liquid form, either as a water soluble solution, or as a solvent based solution, or in solid form, such as a freeze dried or encapsulated powder. The fragrance material may also be sprayed upon the surface of the hydrogel resin. Exemplary of such fragrance materials are such well known fragrance ingredients which may be used to create satisfactory aromas, including but not limited to myrrh, cedarwood, cedrenol, cedrol, birch, methyl salicylate, fir balsam, sandalwood, santalol, juniper, benzoin, coniferyl benzoate, thyme, thymol, bay, eugenol, myrcene, basil, camphor, methyl cinnamate, cinnamon, cinnamic aldehyde, rosemary, clove, and borneol. However, it has been learned that not all forms or types of fragrancing materials are compatible with the preferred gel forming resin, cross linked sodium polyacrylate. Long straight chain hydrocarbon fragrances, such as terpenes (Limonene, for example) form a softer or more flowing gel with sodium polyacrylate. The initial reaction of such long straight chain hydrocarbons may be similar to that of other fragrances, but may result in softening of the gel with passage of time. This type of reaction is undesirable in a do-it-yourself consumer kit, since a soft, and thus flowable gel, is not as aesthetically pleasing as a firm gel, and may pose a potential spillage risk as well. Accordingly, terpene based or other long straight chain hydrocarbon based fragrances are unsuitable for use in the present invention.

Preferably, the fragrance or air freshener is a fragrance comprising one or more volatile organic compounds which are available from perfume suppliers such as Firmenich Inc., Takasago Inc., Noville Inc., Quest Co., International Flavors & Fragrances, and Givaudan-Roure Corp. Most conventional fragrance materials are volatile essential oils. The fragrance may be a synthetically formed material, or a naturally derived oil such as oil of bergamot, caraway, geranium, lavender, origanum, petitgrain, white cedar, patchouli, lavandin, neroli, rose absolute, and the like, excluding terpene or other straight chain hydrocarbon derived fragrances. A fragrance can be relatively simple in composition, or can be a complex mixture of natural and synthetic chemical components. Synthetic types of fragrance compositions may be employed, either alone or in combination with natural oils, as described in U.S. Pat. Nos. 4,324,915; 4,411,829; and 4,434,306. Other artificial liquid fragrances include geraniol, geranyl acetate, isoeugenol, linalool, linalyl acetate, phenethyl alcohol, methyl ethyl ketone, methylionone, isobomyl acetate, and the like.

A liquid fragrance may also be formed into a thixotropic gel by the addition of a thickening agent, such as a cellulosic material, a polymeric thickener, or a fumed silica of the type marketed under the Cabosil trademark by Cabot Corporation. A fragrance ingredient can also be in the form of a crystalline solid, which has the ability to sublime into the vapor phase at ambient temperatures. A crystalline fragrance material can be selected from organic compounds which include vanillin, ethyl vanillin, coumarin, tonalid, calone, heliotropene, musk xylol, cedrol, musk ketone benzophenone, raspberry ketone, methyl naphthyl ketone beta, phenyl ethyl salicylate, veltol, maltol, maple lactone, proeugenol acetate, evemyl, and the like. This type of fragrance can contribute a long term air-treatment capability to an air freshener for use in the present invention. Among the preferred fragrances are such fragrance materials as lilac, jasmine, and rose, and apple type fragrances.

If so desired, the kit may contain a colorant to cause the hydrogel to have a distinct coloration. Such colorant may be present as a separate component to be mixed with the hydrogel resin and the fragrance, or as a pre-mixed component of either. The colorant is preferably present in a water soluble form for even distribution when the component materials are mixed with water to activate the kit, and to form the gel. In the event that a synthetic rubber gel forming resin, such as the aforementioned Dynasorb Terrasafe, is utilized, the coloring agent should be provided in an oil based formulation, such as in an oil based fragrance. In such an event, no water is necessary to form the gel material, and appropriate instruction to the consumer is provided.

The kit may also contain a suitable container for the hydrogel air freshener, or may contain instructions as to the type of container best used for the preparation of the air freshener. The container should be a dish, bowl, jar, or such, of suitable volume and aesthetically pleasing, made of an inactive material, such as glass or plastic which will not react with any of the components of the kit, or with any solvent material present in the fragrance or hydrogel resin. Glass or hard transparent plastic is preferred.

In a preferred embodiment, the kit as sold to the consumer comprises a glass jar, such as commonly used for jar candles, containing a small plastic bag of pre-colored sodium polyacrylate hydrogel resin, and a small glass or plastic vial of fragrance. The kit may further comprise a plastic lid, having small openings therein whereby fragrance may be released to the atmosphere after the hydrogel air freshener kit has been activated by the addition of water by the consumer, and reclosure with the lid to prevent accidental exposure of the contents such as to children touching or playing with the gel. The lid is also designed, such by as having openings cut therein, to enhance air flow for better fragrance delivery to the environment.

When the consumer wishes to avail himself or herself of the air freshener device, the consumer may open the component packages, and mix the contents thereof in the container provided, or in a suitable container of the consumers choice. Thus, the consumer may pre-mix the resin, the colorant if present individually, and the fragrance material. To this mixture, the consumer adds a volume of water in accordance with instructions present with the kit, said volume of water being of a relatively large volume in comparison to the volume of resin, colorant, and fragrance present. After addition of the water, the consumer may stir, mix, or shake the liquid mixture to assure full contact of the water with all of the other components present. Similarly, if the gelling material provided comprises the synthetic rubber superabsorbent resin, an appropriate volume of hydrocarbon oil is provided, either with the fragrancing material, with the coloring material, or as a separate component in its own vial or container, with the appropriate instructions for combination by the consumer. The fully mixed container may then be put aside for a few minutes, for the reaction and absorption of the water or oil and the formation of a gel. As the gel forms, the fragrance and colorant if present are evenly distributed in the gel. Dependent upon the resin used, the particulate size thereof, the volume of water or oil added, and the manner and duration of mixing, the consumer may obtain a variety of gel forms, ranging from a clear gel similar in appearance to a body of liquid, to a slushy mixture, or to container of crystal like, small “cubes” of gel. It is to be noted that no surfactant need be present in the gel resin system of the present invention, and that if a surfactant is present, the resultant gel tends to be cloudy rather than clear.

As an example of the above embodiment of the invention, a hydrogel air freshener kit was prepared, comprising a small plastic container containing 0.5 to about 2 grams of Alcosorb G5 sodium polyacrylate polymer resin, and a small vial containing from about 2 to about 8 grams of a liquid jasmine fragrance. The polyacrylate resin was premixed with about 0.04 weight percent of the coloring agent Red F-5B, from Clairiant. The resin and the fragrance were provided in a small candle jar, having a plastic lid. Upon opening of the lid and removal of the contained components, the resin and fragrance were pre-mixed in the candle jar, followed by addition of about 150 cc of tap water. After about 1.5 minutes, a light red gel was formed, containing the fragrance. Water hardness will have an effect on the gelling time of the mixture, and to compensate for the variation in water hardness anticipated in the market for which the specific kit is prepared, variations in proportion of the components provided may be made. Fragranced gel was left open to the atmosphere, with no cover, and the air freshening effect thereof was noticeable for an extended period of time. The finished gel may be contained in a variety of containers, and for convenience this is achieved by mixing the components with water in the final container desired. The greater the surface area of gel exposed to the air, the stronger the fragrance strength, due to the greater number of fragrance molecules released into the atmosphere. Smaller mouth containers, on the other hand, will have a weaker fragrance strength, due to a lower evaporation rate, but, conversely, a longer product life.

In another embodiment of the invention, a kit is provided to the consumer as set forth above, wherein the hydrogel resin is provided in the form of a sachet, characterized by having one way permeability to water, wherein the colorant and fragrance material are included within the sachet. For example, the fragrance may be provided as a solid particulate material, either crystalline or powder, or as a spray dried fragrance, either mixed with or upon the surface of the resin. Suitable forms of solid fragrance materials include spray dried powders, and encapsulated fragrances. Alternatively, the fragrance may be a liquid, included in a breakable container within the sachet, which the consumer may squeeze or flex to break open and release the fragrance. Upon addition of water to the sachet, a hydrogel rapidly forms, constrained by the packet. It is also possible for the packet to be such that it would be dissolved by the water, or easily torn apart by the expansion of the rapidly formed gel upon reaction of the resin and the water. Such a sachet may be provided with a suitable container into which the necessary amount of water may be poured by the consumer, or, alternatively, by itself for use in a container of the consumer's choice. As previously indicated, the container should be preferably of glass, or an inert plastic material which will not react with or be effected by the water, the resin, the resulting gel, any colorant present, or the fragrance material.

As an example of this embodiment of the invention, a sachet of sodium polyacrylate resin, comprising 0.5 to 2 grams of resin having a particle size between about 150 and 850 microns, was provided in a water insoluble cellulosic fiber packet with a spray dried fragrance absorbed in starch included, packaged in a candle jar as in the above example. The sachet was submerged in sufficient water in the candle jar to provide a water to resin ratio of about 150:1, for about 5 minutes, at which time the water and fragrance had been essentially absorbed by the resin, providing a fragrant gel air freshener. The gel was exposed to the atmosphere, and the fragrance was noticeable for a considerable number of days, during which time the gel dried and shrank. The gel was subsequently subjected to a soaking in additional water to attempt to regenerate the air freshener, which resulted in a regeneration of the gel, but only low notes of the fragrance were noted after removal of the gel from the water.

As an alternative of this embodiment, a water activated dye or coloring agent was printed on the surface of the sachet to serve as an indicator of usage. In this instance the color was vibrant when the sachet was wet, and almost colorless when dry.

In another form of the invention, a pad of material such as an absorbent cotton or other cellulosic or non-woven polyolefin material, may be coated or impregnated with a mixture of resin and fragrance, dried, and provided to the consumer in moisture resistant packaging. Such a pad need not be limited to cellulosic materials, but may include polyolefins, such as polyethylene, or polyesters. This pad may then be removed from the moisture resistant packaging, and activated by spraying with water or immersion in water, to provide a hydrogel air freshener which will release fragrance over a period of time. After activation of the powdered gel, the pad may be placed in a location of the consumer's choice for extended air freshening.

As an example of this embodiment of the invention, a cellulosic pad, comprising paper, was impregnated with polyacrylate polymer powder (Gelling Agent 14) and a spray dried fragrance, in a non-aqueous carrier, dried, and set aside. After drying, the impregnated pad was placed in a beaker containing about ½ cup of water, and allowed to absorb the water and form a thickened pad of air freshener. The pad was then hung from a convenient location, and provided air freshening for 10 to 30 days.

In still another form of the invention, a pad was coated with resin, with no fragrance, and dried. The pad was then placed in a small bowl, open to a controlled flow of water containing a water-soluble fragrance. Alternatively, the bowl was attached to a reservoir of fragrance solution having a controlled release of solution equivalent to the evaporation rate of the liquid absorbed into the pad or hydrogel. This form of air freshener was found to have a life span determined by the volume of water and fragrance available. Since the hydrogel was not activated to form a gel until it was first contacted with the water, this pad provided a source of consistent fragrancing which was capable of being stored for an extended period of time, and economically shipped, since there was no liquid present. Further, since the fragrance being emitted to the atmosphere was replenished by the addition of fresh fragrance-containing water, at the rate of fragrance evaporation, the air freshener provided consistent and fresh fragrance for as long a period as the source of liquid was available. Hence, the consumer could purchase separate, and perhaps differing, containers of water and fragrance, and continue to obtain an air freshening effect indefinitely, by merely replacing the container of fragrance as it emptied.

These resin impregnated or coated pads were found to constitute very economical forms of air freshener devices, whether the fragrance was present in the pad, or in the liquid with which the pad was subsequently contacted by the consumer. Due to the fact that there was no liquid present in the pad, shipping costs were minimized. This form of packaging also permits great flexibility in terms of the product offered the consumer. By packaging the pad with a container for controlled contact with water, i.e. a metering bottle and tray into which the content of the water may be metered, and in which the pad may be located, the consumer may receive a totally dry system which may be opened, easily assembled, and activated by addition of water (or fragrance solution, packaged separately) to the metering bottle. In an alternate embodiment, resin impregnated pads may be sold separately, having various fragrances therein, so as to offer the consumer replacement pads of varying fragrance to be used with a reusable metering bottle and tray. Or, various solutions of differing fragrance may be offered to be utilized with gel impregnated pads (having no fragrance), so as to permit the consumer the opportunity to vary the fragrance as desired.

### INDUSTRIAL APPLICABILITY

While the present invention has been described with respect to what is at present considered to be the preferred embodiments, it is to be understood that the invention is not to be limited to the disclosed embodiments. To the contrary, the invention is intended to cover various modifications and equivalent arrangements within the spirit and scope of the appended claims. The scope of the following claims is to be accorded the broadest interpretation so as to encompass all such modifications and equivalent formulations and functions.

1) A kit for the preparation, by a consumer, of a fragranced hydrogel air freshener by the addition of a gel forming liquid to a gel forming resin and a fragrance in a container, said kit comprising a package containing a measured quantity of a superabsorbent gel forming resin having a particle size distribution between about 100 and 900 microns, said resin selected from the group consisting of cross linked sodium polyacrylate, potassium polyacrylate/acrylamide copolymer, polyacrylamide polymer, and synthetic rubber based superabsorbent, and a package containing a measured quantity of fragrance material.

2) The kit of claim 1, additionally comprising a container in which said measured quantities of resin and fragrance material may be mixed with a gel forming liquid.

3) the kit of claim 2, wherein said gel forming resin comprises cross linked sodium polyacrylate polymer.

4) The kit of claim 3, further comprising a coloring agent.

5) The kit of claim 3, wherein said fragrance is a liquid fragrance.

6) The kit of claim 3, wherein said fragrance is a liquid enclosed within a breakable container.

7) The kit of claim 2, wherein said resin and said fragrance are both solid.

8) The kit of claim 1, wherein said resin and said fragrance are provided in a sachet.

9) The kit of claim 8, wherein said sachet contains particulate sodium polyacrylate polymer and a fragrance material, and said sachet is characterized by one way permeability to water.

10) The kit of claim 8, further comprising a coloring agent.

11) A kit for the preparation, by the consumer, of a fragranced hydrogel air freshener, said kit comprising a pad impregnated with sodium polyacrylate superabsorbent gel forming resin, said kit being activated by the controlled addition thereto of a water solution of fragrance.

12) A kit as set forth in claim 11, further comprising a container for providing a controlled flow of said water solution to said pad so as to provide a consistent dispensing of fragrance.

13) A kit as set forth in claim 12, wherein said pad is a replaceable element of said kit.

14) A kit for the preparation, by the consumer, of a fragranced hydrogel air freshener, said kit comprising a package containing a pad impregnated with sodium polyacrylate superabsorbent gel forming resin and a fragrance, said kit being activated by the controlled addition thereto of water.

15) A kit as set forth in claim 14, further comprising a container for providing a controlled flow of said water to said pad so as to provide a consistent dispensing of fragrance.

16) A kit as set forth in claim 15, wherein said pad is a replaceable element of said kit.

17) A kit for the preparation, by a consumer, of a fragranced hydrogel air freshener by the addition of a gel forming liquid to a gel forming resin and a fragrance in a container, said kit comprising a package containing a measured quantity of a superabsorbent gel forming synthetic rubber resin having a particle size distribution between about 100 and 900 microns, and a package containing a measured quantity of fragrance material.
